(19) United States
(12) Patent Application Publication
(10) Pub. No.: US 2024/0341886 A1
(43) Pub. Date: Oct. 17, 2024

(54) SURGICAL ROBOTIC SYSTEM AND SURGICAL INSTRUMENT THEREOF

(71) Applicant: Covidien LP, Mansfield, MA (US)

(72) Inventor: Brock Kopp, Colorado Springs, CO (US)

(21) Appl. No.: 18/294,769

(22) PCT Filed: Aug. 18, 2022
(86) PCT No.: PCT/IB2022/057760

§ 371 Date: Feb. 2, 2024

Related U.S. Application Data
(60) Provisional application No. 63/237,230, filed on Aug. 26, 2021.

(51) Int. Cl. A61B 34/00; A61B 17/29; A61B 34/35

(52) U.S. Cl. CPC A61B 34/71; A61B 17/29; A61B 34/35; A61B 2034/715

(57) ABSTRACT

A surgical instrument is configured for coupling with an instrument drive unit that drives an actuation of the surgical instrument and operatively supports the surgical instrument. The surgical instrument includes a housing, an elongate body extending distally from the housing, an end effector extending distally from the elongate body, and a plurality of cables for performing a plurality of functions of the end effector. The elongate body is rotatable relative to the housing to adjust a rotational orientation of the end effector. The elongate body is also coupled to the housing such that a rotation of the elongate body relative to the housing results in a translation of the elongate body to adjust a tension in the cables.

### BACKGROUND

Surgical robotic systems have been used in minimally invasive medical procedures. Some surgical robotic systems include a console supporting a robot arm and a surgical instrument or at least one end effector that includes forceps or a grasping tool that is mounted to the robot arm. The robot arm provides mechanical power to the surgical instrument for its operation and movement. Each robot arm may include an instrument drive unit that is operatively connected to the surgical instrument.

Prior to or during use of the robotic system, surgical instruments are selected and connected to the instrument drive units of each robot arm. Cables for actuating functions of the surgical instrument can lose their tension from the normal wear and tear during their usage.

### SUMMARY

In accordance with an aspect of the disclosure, a surgical instrument for use in a surgical robotic system is provided. The surgical instrument includes an outer housing and a shaft. The outer housing is configured to be engaged with an instrument drive unit. The shaft has a proximal end portion rotationally coupled to a distal end portion of the outer housing, and a distal end portion configured to be coupled to an end effector. The shaft is configured to move along a longitudinal axis defined by the shaft relative to the outer housing in response to a rotation of the shaft relative to the outer housing.

In aspects, the proximal end portion of the shaft may be threadedly coupled to the distal end portion of the outer housing.

In aspects, the proximal end portion of the shaft may have a threaded outer surface, and the distal end portion of the outer housing may have a threaded inner surface coupled to the threaded outer surface of the shaft. The threaded inner surface of the outer housing may define a channel through which the shaft extends.

In aspects, the surgical instrument may further include a cable having a distal end portion coupled to the end effector such that a tension in the cable is adjusted as the shaft moves along the longitudinal axis thereof relative to the outer housing.

In aspects, the surgical instrument may further include a drive screw and a drive unit supported on the drive screw. The drive screw may be rotatably supported in the outer housing and may be configured to be drivingly coupled to an instrument drive unit. The drive nut may have a proximal end portion of the cable fixed thereto. The drive nut may be configured to translate relative to the drive screw in response to a rotation of the drive screw to translate the cable.

In aspects, cable termination within the housing may be to capstans. These capstans may replace the drive screw/drive nut configuration within the housing to transmit motion from the IDU coupler to the cable loop.

In aspects, cable may extend longitudinally through the shaft and may have a proximal end portion fixed within the outer housing. The distal end portion of the cable may be fixed to the end effector.

In accordance with an aspect of the disclosure, a surgical instrument for use with a surgical robotic system is provided that includes an outer housing configured to be operably engaged with an instrument drive unit, a shaft extending distally from the outer housing and having a proximal end portion rotationally coupled to the outer housing, an end effector coupled to a distal end portion of the shaft, and a cable extending between the outer housing and the end effector. The cable is configured to perform a function of the end effector and the shaft is configured to move axially relative to the outer housing in response to a rotation of the shaft relative to the outer housing to adjust a tension in the at least one cable.

In aspects, the proximal end portion of the shaft may be threadedly coupled to the outer housing.

In aspects, the proximal end portion of the shaft may have a threaded surface, and the outer housing may have a threaded surface coupled to the threaded surface of the shaft.

In aspects, the threaded surface of the outer housing may define a channel through which the proximal end portion of the shaft extends.

In aspects, the cable may have a proximal end portion disposed within the outer housing, and a distal end portion coupled to the end effector.

In aspects, the surgical instrument may further include a rotatable drive rod operably coupled to the proximal end portion of the shaft. The shaft may be configured to rotate in response to a rotation of the drive rod.

In accordance with an aspect of the disclosure, a surgical robotic system is provided that includes a surgical robotic arm, an instrument drive unit configured to be supported on the surgical robotic arm, and a surgical instrument. The surgical instrument includes an outer housing configured to be operably engaged with the instrument drive unit, a shaft extending distally from the outer housing and having a proximal end portion rotationally coupled to the outer housing, an end effector coupled to a distal end portion of the shaft, and a cable extending between the outer housing and the end effector. The cable is configured to perform a function of the end effector and shaft is configured to move axially relative to the outer housing in response to a rotation of the shaft relative to the outer housing by an actuation of the instrument drive unit to adjust a tension in the cable.

In aspects, the surgical instrument may further include a rotatable drive rod having a proximal end portion configured to be operably coupled to the instrument drive unit, and a distal end portion operably coupled to the proximal end portion of the shaft. The shaft may be configured to rotate in response to a rotation of the drive rod.

Further details and aspects of exemplary embodiments of the disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Embodiments of the disclosure are described herein with reference to the accompanying drawings, wherein:

FIG. 1 is a schematic illustration of a surgical robotic system in accordance with the disclosure;

FIG. 2 is a top, perspective view illustrating a surgical instrument of the surgical robotic system of FIG. 1;

FIG. 3 illustrates a cross-section, taken alone line 3-3 in FIG. 2, of the surgical instrument; and

FIG. 4 is a schematic illustration of the surgical instrument of FIG. 2 with the end effector in a detached state from a shaft of the surgical instrument.

### DETAILED DESCRIPTION

Embodiments of the disclosed surgical robotic system including an instrument drive unit and a surgical instrument and methods thereof are described in detail with reference to the drawings, in which like reference numerals designate identical or corresponding elements in each of the several views. As used herein the term “distal” refers to that portion of the surgical instrument and/or instrument drive unit that is closer to the patient, while the term “proximal” refers to that portion of the surgical instrument and/or instrument drive unit that is farther from the patient.

Cable-driven robotic instruments may have issues with cable tension relaxing over time. This may happen with both metallic cables, due to wear/construction stretch, and with polymer cables, which may slowly creep/extend over time, and thus lose tension. Some means for tensioning cables involve using a separate drive mechanism, which occupies space within the surgical instrument.

Accordingly, the disclosure uses an existing degree of freedom to simultaneously tighten (or loosen) cables. More specifically, the degree of freedom and tension are coupled, but the mechanism allows sufficient compliance such that the degree of freedom may be actuated without adversely effecting tension in the cables. However, driving beyond the normal degree of freedom's range of motion does meaningfully adjust the cable tension.

In one embodiment, the surgical instrument includes a threaded interface between the instrument shaft and the instrument housing. The threaded interface has a relatively fine threading so that the user may roll the instrument shaft (e.g., rotate the instrument shaft about its longitudinal axis) across a typical range of motion during use, but while the clinician is not performing a surgical procedure, or during a surgical procedure but while the end effector is outside of the patient, the surgical robotic system may automatically rotate the instrument shaft multiple times to increase/decrease tension in the cables. In aspects, a spring-loaded mechanism may be included in the cable routing to add compliance, which would allow the thread pitch to be reasonable/manufacturable while also allowing the clinician to rotate the shaft through the desired range of motion.

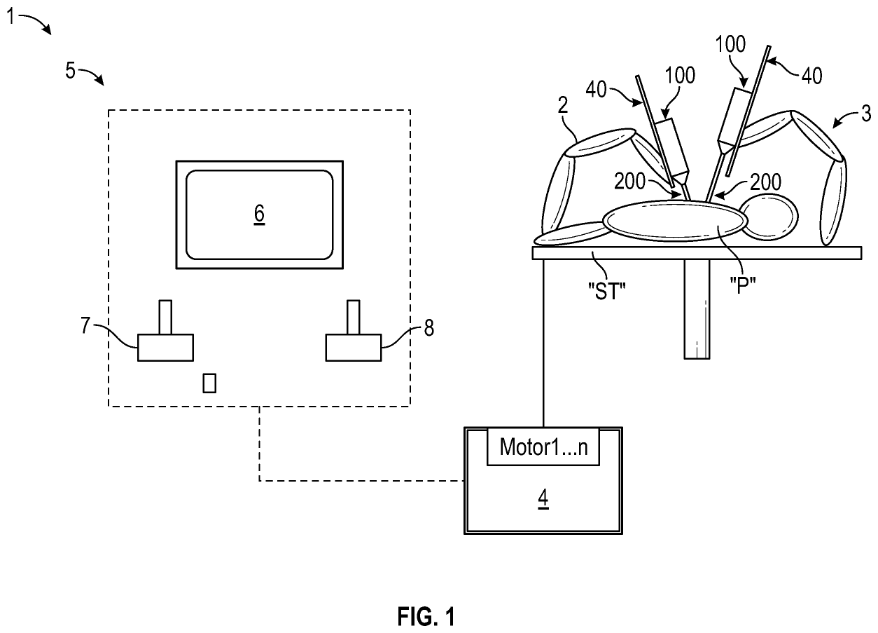

Referring initially to FIG. 1, a surgical system, such as, for example, a surgical robotic system is shown generally as surgical system 1 and generally includes a plurality of robotic arms 2, 3; a control device 4; and an operating console 5 coupled with control device 4. Operating console 5 includes a display device 6, which is set up in particular to display three-dimensional images; and manual input devices 7, 8, by means of which a person (not shown), for example a surgeon, is able to telemanipulate robotic arms 2, 3 in a first operating mode, as known in principle to a person skilled in the art.

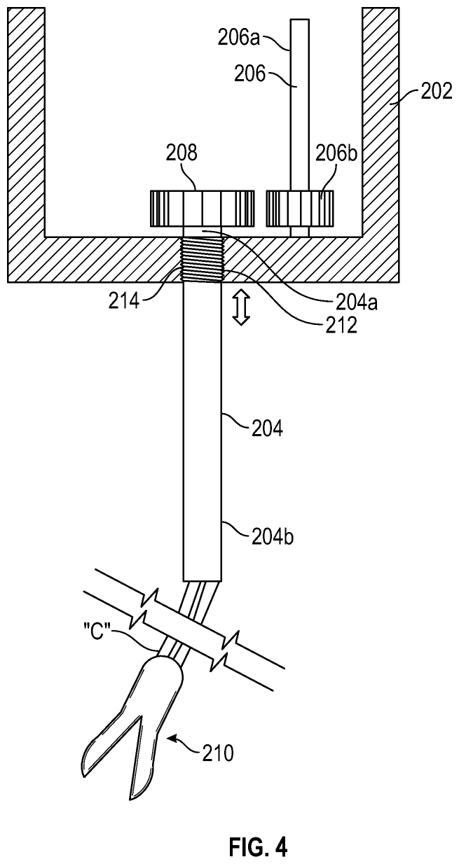

Each of the robotic arms 2, 3 is composed of a plurality of members, which are connected through joints. System 1 also includes an instrument drive unit 100 connected to distal ends of each of robotic arms 2, 3. A surgical instrument 200 supporting an end effector 210 (FIG. 4) may be attached to instrument drive unit 100, in accordance with any one of several embodiments disclosed herein, as will be described in greater detail below.

Robotic arms 2, 3 may be driven by electric drives (not shown) that are connected to control device 4. Control device 4 (e.g., a computer) is set up to activate the drives, in particular by means of a computer program, in such a way that robotic arms 2, 3, their instrument drive units 100 and thus the surgical instrument 200 (including end effector 210) execute a desired movement according to a movement defined by means of manual input devices 7, 8. Control device 4 may also be set up in such a way that it regulates the movement of robotic arms 2, 3 and/or of the drives.

Surgical system 1 is configured for use on a patient “P” lying on a surgical table “ST” to be treated in a minimally invasive manner by means of an end effector. Surgical robotic system 1 may also include more than two robotic arms 2, 3, the additional robotic arms likewise being connected to control device 4 and being telemanipulatable by means of operating console 5. The surgical instrument 200 (including end effector 210) may also be attached to the additional robotic arm.

The surgical robotic system 1 may further include a surgical instrument holder (not explicitly shown) configured to be coupled with or to the robotic arm 2, 3. The surgical instrument holder holds the instrument drive unit 100 and the surgical instrument 200. The surgical instrument holder supports or houses a motor (not explicitly shown), which receives controls and power from the control device 4 to effect a rotation of an inner motor pack (not explicitly shown) of the instrument drive unit 100, which results in a rotation of the surgical instrument 200 about a longitudinal axis thereof. The surgical instrument holder may be slidably mounted onto a rail 40 of the robotic arm 2, 3 and moved along the rail 40 via a motor driven chain or belt or the like to adjust a position of the surgical instrument 100.

Reference may be made to U.S. Pat. No. 8,828,023, filed on Nov. 3, 2011, entitled “Medical Workstation,” the entire content of which is incorporated herein by reference, for a detailed discussion of the construction and operation of surgical system 1.

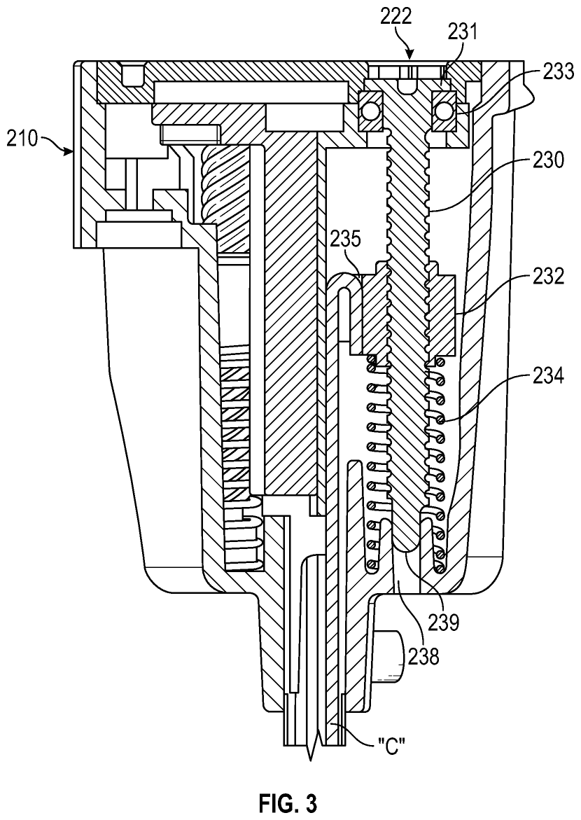

Control device 4 may control a plurality of motors (Motor 1 . . . n) with each motor configured to drive a pushing or a pulling of a cable “C” (FIGS. 3 and 4) extending between end effector 210 of surgical instrument 200 and a respective driven member (i.e., a first drive nut 232 of FIG. 4) of surgical instrument 400. In use, as cables “C” are pushed or pulled, cables “C” effect operation and/or movement of the end effector 210 of the surgical instrument 200. It is contemplated that control device 4 coordinates the activation of the various motors (Motor 1 . . . n) to coordinate a pushing or a pulling motion of a respective cable “C” to coordinate an operation and/or movement of a respective end effector 210. In embodiments, each motor can be configured to actuate a drive rod or a lever arm to effect operation and/or movement of each end effector of surgical instrument 200.

Instrument drive unit 100 is configured for driving an actuation of end effector 210 of surgical instrument 200 and to operatively support surgical instrument 200 therein. Instrument drive unit 100 transfers power and actuation forces from motors to surgical instrument 200 to ultimately drive movement of the cables “C” that are attached to end effector 210.

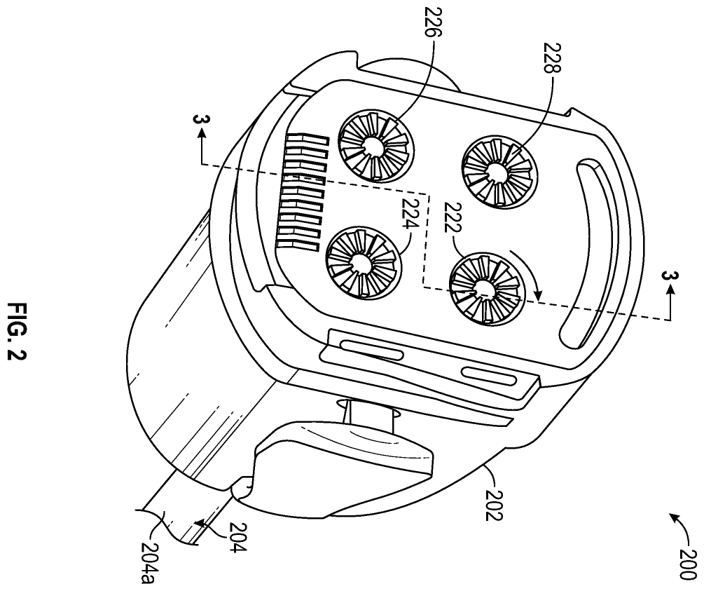

With reference to FIGS. 2 and 3, surgical instrument 200 generally has an outer housing 202 configured to be engaged with instrument drive unit 100 and a shaft 204, such as, for example, an elongate tube engaged with outer housing 202 and extending distally therefrom. Shaft 204 has a proximal end portion 204a coupled to instrument drive unit 100, and a distal end portion 204b (FIG. 4). End effector 210 extends distally from the distal end portion 204b of shaft 204 and is configured for performing a plurality of surgical functions.

The surgical instrument 200 has a plurality of motor interfaces 222, 224, 226, 228 (FIG. 2) rotatably supported by the outer housing 202 and configured to be operably coupled to a corresponding drive member of the instrument drive unit 100. The surgical instrument 200 further includes a drive screw 230, a first drive nut 232, a first spring 234, and a first cable “C.” The first drive screw 230 includes a first head 231 and a distal nub 239. The first head 231 may have radial teeth, a slot, a female connector, a male connector, or any suitable interface for coupling coaxial rotating shafts such that the first head 231 is configured to mechanically couple the first drive screw 230 to a motor of the instrument drive unit 100. The first drive screw 230 is supported within the outer housing 202 by a first bearing 233 positioned adjacent the first head 231. The distal nub 239 is received within an opening 238 defined by the outer housing 202 such that the first bearing 233 and the distal nub 239 support the first drive screw 230 within the outer housing 202 and enable the first drive screw 230 to rotate about its longitudinal axis, maintain the longitudinal axis of the first drive screw 230 parallel to a longitudinal axis of the elongate shaft 204, and prevent the first drive screw 230 from translating along its longitudinal axis.

The first drive nut 232 is disposed over a threaded portion of the first drive screw 230 such that the first drive nut 232 and the first drive screw 230 are threadably coupled with one another. Specifically, as the first drive screw 230 is rotated in a first direction, e.g., clockwise rotation about the longitudinal axis of the drive screw 230, the first drive nut 232 translates proximally along the first drive screw 230 towards the first head 231 and when the first drive screw 230 is rotated in a second direction opposite the first direction, e.g., counter-clockwise, the first drive nut 232 is translated distally along the first drive screw 230 away from the first head 231. The first drive nut 232 defines a first slot 235 that receives a portion of the first cable “C” such that as the first drive nut 232 translates along the first drive screw 230, the first cable “C” cooperates with translation of the first drive nut 232. Specifically, the first cable “C” is retracted as the first drive nut 232 translates proximally and the first cable “C” is relaxed as the first drive nut 232 translates distally.

With reference to FIG. 4, the surgical instrument 200 further includes a rotatable drive rod 206 rotatably supported in the outer housing 202. The drive rod 206 has a proximal end portion 206a configured to be operably coupled to a motor of the instrument drive unit 100 (FIG. 1) such that the drive rod 206 rotates in response to an actuation by the instrument drive unit 100. The drive rod 206 has a distal end portion 206b having a drive gear operably coupled to a driven gear 208 of the shaft 204. As such, the shaft 204 is configured to rotate about a longitudinal axis thereof in response to a rotation of the drive rod 206. In aspects, the drive rod 206 and the driven gear 208 may be a belt/pulley connection.

The shaft 204 has the cables “C” extending therethrough to allow the cables “C” to connect to the end effector 210 to control movement of the end effector 210 in three degrees-of-freedom (DOF), e.g., yaw, pitch, and roll, and/or to control various operations of the end effector 210. The proximal end portion 204a of the shaft 204 is configured to move axially relative to the outer housing 202 in response to a rotation of the shaft 204 relative to the outer housing 202 to adjust a tension in the cables “C.”

For example, the proximal end portion 204a of the shaft 204 has a threaded outer surface 212, and the outer housing 202 has a threaded inner surface 214 coupled to the threaded outer surface 212 of the shaft 204. The threaded inner surface 214 of the outer housing 202 defines a channel 216 through which the proximal end portion 204a of the shaft 204 extends. The thread pitch of the threaded outer surface 212 of the shaft 204 is such that a rotation of the shaft 204 relative to the outer housing 202 in a normal operating range (e.g., between about 0 degrees and about 360 degrees) translates the shaft 204 a negligible amount not sufficient to meaningfully adjust the tension in the cables “C.” For example, the threaded inner surface 214 may have a 12 mm×0.5 mm thread pitch.

However, a rotation of the shaft 204 greater than 360 degrees, such as, for example, about 720 degrees or more, does meaningfully adjust the tension in the cables “C.”

In operation, to rotate the end effector 210 to adjust the roll of the end effector 210, the instrument drive unit 100 is actuated to rotate the drive rod 206 of the surgical instrument 200 whereby the drive rod 206 rotates the shaft 204. Since the end effector 210 is non-rotatably attached to the distal end portion 204b of the shaft 204, the end effector 210 rotates with the shaft 204 about the longitudinal axis of the shaft 204.

To adjust a tension in the cables “C” of the surgical instrument 200, the instrument drive unit 100 may be actuated to rotate the drive rod 206 of the surgical instrument 200 to cause the shaft 204 to rotate more than 360 degrees, such as, for example, at least 720 degrees. Due to the threaded connection between the proximal end portion 204a of the shaft 204 and the outer housing 202, rotation of the shaft 204 relative to the outer housing 202 drives a translation (e.g., proximal or distal) of the shaft 204 and the attached end effector 210 relative to the outer housing 202. As the shaft 204, along with the end effector 210, translate relative to the outer housing 202, the cables “C” are either tensioned or slackened due to the cables “C” being fixed between the end effector 210 and the outer housing 202. Re-tensioning of the cables “C” may be needed if and when the cables ‘C” slacken over time. It may be desirable to intentionally decrease the tension in the cables “C” during an emergency to facilitate a manual override.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A surgical instrument for use with a surgical robotic system, the surgical instrument comprising:
an outer housing configured to be engaged with an instrument drive unit; and
a shaft having a proximal end portion rotationally coupled to a distal end portion of the outer housing, and a distal end portion configured to be coupled to an end effector, wherein the shaft is configured to move along a longitudinal axis defined by the shaft relative to the outer housing in response to a rotation of the shaft relative to the outer housing.

2. The surgical instrument according to claim 1, wherein the proximal end portion of the shaft is threadedly coupled to the distal end portion of the outer housing.

3. The surgical instrument according to claim 2, wherein the proximal end portion of the shaft has a threaded outer surface, and the distal end portion of the outer housing has a threaded inner surface coupled to the threaded outer surface of the shaft, the threaded inner surface of the outer housing defines a channel through which the shaft extends.

4. The surgical instrument according to claim 1, further comprising at least one cable having a distal end portion coupled to the end effector such that a tension in the at least one cable is adjusted as the shaft moves along the longitudinal axis thereof relative to the outer housing.

5. The surgical instrument according to claim 4, further comprising:
a drive screw rotatably supported in the outer housing and configured to be drivingly coupled to an instrument drive unit; and
a drive nut supported on the drive screw and having a proximal end portion of the at least one cable fixed thereto, wherein the drive nut is configured to translate relative to the drive screw in response to a rotation of the drive screw to translate the at least one cable.

6. The surgical instrument according to claim 4, wherein the at least one cable extends longitudinally through the shaft and has a proximal end portion fixed within the outer housing, the distal end portion of the at least one cable being fixed to the end effector.

7. A surgical instrument for use with a surgical robotic system, the surgical instrument comprising:
an outer housing configured to be operably engaged with an instrument drive unit;
a shaft extending distally from the outer housing and having a proximal end portion rotationally coupled to the outer housing;
an end effector coupled to a distal end portion of the shaft; and
at least one cable extending between the outer housing and the end effector, the at least one cable configured to perform at least one function of the end effector, wherein the shaft is configured to move axially relative to the outer housing in response to a rotation of the shaft relative to the outer housing to adjust a tension in the at least one cable.

8. The surgical instrument according to claim 7, wherein the proximal end portion of the shaft is threadedly coupled to the outer housing.

9. The surgical instrument according to claim 7, wherein the proximal end portion of the shaft has a threaded surface, and the outer housing has a threaded surface coupled to the threaded surface of the shaft.

10. The surgical instrument according to claim 9, wherein the threaded surface of the outer housing defines a channel through which the proximal end portion of the shaft extends.

11. The surgical instrument according to claim 7, wherein the at least one cable has a proximal end portion disposed within the outer housing, and a distal end portion coupled to the end effector.

12. The surgical instrument according to claim 7, further comprising:
a drive screw rotatably supported in the outer housing and configured to be drivingly coupled to an instrument drive unit; and
a drive nut supported on the drive screw and having a proximal end portion of the at least one cable fixed thereto, wherein the drive nut is configured to translate relative to the drive screw in response to a rotation of the drive screw to translate the at least one cable.

13. The surgical instrument according to claim 12, wherein the at least one cable extends longitudinally through the shaft and has a proximal end portion fixed within the outer housing, the distal end portion of the at least one cable being fixed to the end effector.

14. The surgical instrument according to claim 7, further comprising a rotatable drive rod operably coupled to the proximal end portion of the shaft, wherein the shaft is configured to rotate in response to a rotation of the rotatable drive rod.

15. A surgical robotic system, comprising:
a surgical robotic arm;
an instrument drive unit configured to be supported on the surgical robotic arm; and
a surgical instrument including:
an outer housing configured to be operably engaged with the instrument drive unit;
a shaft extending distally from the outer housing and having a proximal end portion rotationally coupled to the outer housing;
an end effector coupled to a distal end portion of the shaft; and
at least one cable extending between the outer housing and the end effector, the at least one cable configured to perform at least one function of the end effector, wherein the shaft is configured to move axially relative to the outer housing in response to a rotation of the shaft relative to the outer housing by an actuation of the instrument drive unit to adjust a tension in the at least one cable.

16. The surgical robotic system according to claim 15, wherein the proximal end portion of the shaft is threadedly coupled to the outer housing.

17. The surgical robotic system according to claim 15, wherein the proximal end portion of the shaft has a threaded outer surface, and the outer housing has a threaded inner surface coupled to the threaded outer surface of the shaft.

18. The surgical robotic system according to claim 15, wherein the surgical instrument further includes:
a drive screw rotatably supported in the outer housing and configured to be drivingly coupled to the instrument drive unit; and
a drive nut supported on the drive screw and having a proximal end portion of the at least one cable fixed thereto, wherein the drive nut is configured to translate relative to the drive screw in response to a rotation of the drive screw to translate the at least one cable.

19. The surgical robotic system according to claim 18, wherein the at least one cable extends longitudinally through the shaft and has a proximal end portion fixed within the outer housing, the distal end portion of the at least one cable being fixed to the end effector.

20. The surgical robotic system according to claim 19, wherein the surgical instrument further includes a rotatable drive rod having a proximal end portion configured to be operably coupled to the instrument drive unit, and a distal end portion operably coupled to the proximal end portion of the shaft, wherein the shaft is configured to rotate in response to a rotation of the rotatable drive rod.
